(19) United States
(12) Patent Application Publication
(10) Pub. No.: US 2015/0182699 A1
(43) Pub. Date: Jul. 2, 2015

(54) SYRINGE EXTENSION

(71) Applicants: Lior RADAY, (US); David DAILY, (US)

(72) Inventors: David Daily, Herzlia (IL); Lior Raday, Kibbutz Bror Hail (IL)

(73) Assignee: DALI Medical Devices Ltd, Yavne (IL)

(21) Appl. No.: 14/412,678

(22) PCT Filed: Jul. 1, 2013
(86) PCT No.: PCT/IB2013/055379

§ 371 Date: Jan. 7, 2015

Related U.S. Application Data
(60) Provisional application No. 61/668,036, filed on Jul. 5, 2012.

(51) Int. Cl. A61M 5/178; A61J 1/20; A61M 39/22; A61J 1/14

(52) U.S. Cl. CPC A61M 5/1782; A61J 1/1406; A61J 1/1475; A61J 1/2096; A61M 39/223; A61J 2001/1481; A61J 2001/201; A61J 2001/2013; A61J 2001/2062

(57) ABSTRACT

Disclosed is an extension assembly for a syringe barrel. The assembly includes a housing having a side wall, a first end, a second end, and a coupling extending from the first end. The coupling is configured to attach to a syringe barrel. The assembly additionally includes a curved fluid channel extending from the coupling to a filling port. The filling port extends from the side wall and is configured to receive a vial which is selectively in fluid communication with the syringe barrel via the curved fluid channel.

### FIELD AND BACKGROUND OF THE INVENTION

The present invention, in some embodiments thereof, relates to a syringe barrel extension, more particularly, but not exclusively, to a syringe barrel extension including a rotatable curved fluid channel selectively extending between a syringe barrel, a filling port, and a needle.

Drugs, vaccines, medicaments, solutions, and sterile fluids such as sterile saline, hereinafter “medication”, are often stored in a sealed vial prior to being injected into a patient. Dry medication, for example medication in a powdered form, may be reconstituted with a fluid prior to injection.

A conventional vial for storing medication has an open end, a radial rim surrounding the open end, a planar rim portion that overlies the vial rim, and a reduced diameter neck portion adjacent the rim. Commonly, such vials are closed by an elastomeric stopper, or other pierceable closure, which is pierced by a needle, typically a syringe needle.

The syringe plunger is partially withdrawn from the barrel of the syringe to fill the syringe with the medication. The needle is then withdrawn from the vial and the syringe is ready to dispense the medication to a patient.

WO2009060419 filed 10 Nov. 2008, the contents of which is hereby incorporated by reference as if fully set forth herein, discloses an adaptor to aid in the activity of transferring fluid from a first container, such as a conventional medical vial having a pierceable closure or stopper, and a second container, such as an injection device (hereinafter also referred to as an “injector”).

US 20070118081 filed 24 May 2007, the contents of which is hereby incorporated by reference as if fully set forth herein, discloses an injector including a retractable needle guard wherein displacement of the needle guard is operative to allow a needle to assume, from a non-penetration position, a penetration position.

Additional Background Applications include:
US 20090018506, filed 15 Mar. 2005;
WO 05025637, filed 15 Sep. 2004;
WO 2008047372A2, filed 24 Apr. 2008; and US 2003/0036725, filed 20 Sep. 2001,
   the contents of all of the above documents are incorporated by reference as if fully set forth herein.

### SUMMARY OF THE INVENTION

According to an aspect of some embodiments of the present invention there is provided an extension assembly for a syringe barrel. The assembly includes a housing having a side wall, a first end, a second end, and a coupling extending from the first end. The coupling is configured to attach to a syringe barrel. The assembly additionally includes a curved fluid channel extending from the coupling to a filling port. The filling port extends from the side wall and is configured to receive a vial which is selectively in fluid communication with the syringe barrel via the curved fluid channel.

In some embodiments of the invention, the assembly includes at least one first resilient element located within the housing, and at least one needle bearing element adapted, when actuated, to be displaced by the at least one first resilient element with respect to the housing element from a non-penetration position to a penetration position.

In some embodiments of the invention, the vial includes a medication.

In some embodiments of the invention, the coupling is configured to attach to the syringe barrel in at least one of: removably, and irremovably.

In some embodiments of the invention, the filling port extends at 90 degrees to the longitudinal axis of the housing.

In some embodiments of the invention, the second end of the housing includes a needle extending therefrom.

In some embodiments of the invention, the filling port is operatively associated with a fluid valve having at least two positions in the assembled configuration: at least one first position in which the vial is in fluid communication with the syringe barrel, and at least one second position in which the needle is in fluid communication with the syringe barrel.

In some embodiments of the invention, when the fluid valve is in the at least one first position, the fluid valve maintains a slidable needle protecting sleeve in a position that operatively surrounds the needle.

In some embodiments of the invention, when the fluid valve is in the at least one first position, a plunger on the syringe barrel may be withdrawn to cause fluid within the vial to enter the syringe barrel.

In some embodiments of the invention, when the fluid valve is in the at least one second position, a slidable needle protecting sleeve is allowed to slide away from its position that operatively surrounds the needle.

In some embodiments of the invention, the fluid includes at least one of: a liquid, a gas, and a powder.

In some embodiments of the invention, when the fluid valve is in the at least one first position, a plunger on the syringe may be depressed to cause fluid within the syringe barrel to enter the vial.

In some embodiments of the invention, the syringe barrel contains at least one of: a liquid, and a gas.

In some embodiments of the invention, the vial contains at least one of: a liquid, a gas, and a powder.

In some embodiments of the invention, the first end of the housing includes a retractable sleeve, the sleeve having at least two positions: at least one first position in which the sleeve surrounds the needle, and at least one second position in which the sleeve at least partially retracts with respect to the first end, thereby exposing at least a portion of the needle, wherein such exposing allows the needle to pierce a substrate.

In some embodiments of the invention, the assembly includes at least one second resilient element located within the housing and operatively associated with the retractable sleeve such that following removal of the assembly from the substrate, the at least one second resilient element extends to press the retractable sleeve forward to at least one third position, beyond the forward extent of the needle.

In some embodiments of the invention, the one second resilient element causes the retractable sleeve to lock in place in said at least one third position.

In some embodiments of the invention, the filling port includes a vial-piercing member configured to pierce the vial such that the piercing facilitates fluid communication with the syringe barrel.

According to another aspect of some embodiments of the present invention there is provided an extension assembly for a syringe barrel. The assembly includes: a housing having a side wall, a first end and a second end. The assembly further includes a coupling extending from the first end, the coupling configured to attach to a syringe barrel, a needle extending from the second end, and a filling port extending from the side wall, the filling port configured to receive a vial. The assembly further includes one fluid valve operatively associated with the filling port, the one fluid valve having at least two positions: at least one first position in which the vial is in fluid communication with the syringe barrel, at least one second position in which the needle is in fluid communication with the syringe barrel.

In some embodiments of the invention, the fluid valve includes an arrow such that in the first position the arrow points to the vial and in the second position, the arrow points to the needle.

According to still another aspect of some embodiments of the present invention there is provided an extension assembly for a syringe barrel. The assembly includes a housing having a side wall, a first end and a second end, and a coupling extending from the first end, the coupling configured to attach to a syringe barrel. The assembly further includes a needle extending from the second end, a needle guard surrounding the needle. Additionally, the assembly includes a filling port extending from the side wall, the filling port configured to receive a vial, and one fluid valve operatively associated with the filling port, the one fluid valve having at least two positions: at least one first position in which at least a portion of the fluid valve contacts the needle guard such that the needle guard is prevented from sliding from a position surrounding the needle and at least one second position in which the at least a portion of the fluid valve is removed from the needle guard such that the needle guard is slidable from the position surrounding the needle.

According to a further aspect of some embodiments of the present invention there is provided a method for delivering at least one medication to a subject, the method including: providing a syringe extension having a rotatable curved fluid channel, attaching a syringe barrel to a first end of the rotatable curved fluid channel and attaching a vial to a second end of the rotatable curved fluid channel. The method further includes establishing fluid communication between the vial and the syringe barrel, drawing a fluid into the syringe barrel, rotating the rotatable curved fluid channel to fluidly communicate between the syringe barrel and a hypodermic needle, inserting the hypodermic needle into a subject, and ejecting the fluid from the syringe barrel into the subject.

In some embodiments of the invention, the vial contains a first medication and the method includes passing a fluid from the syringe barrel into the vial, mixing the contents of the fluid with the first medication and withdrawing the mixture into the syringe barrel.

In some embodiments of the invention, the vial contains a fluid and includes withdrawing the fluid into a syringe barrel containing a medication and mixing the medication with the fluid in the syringe barrel.

In some embodiments of the invention, a first fluid is withdrawn from a first vial into the syringe barrel following which a second fluid is withdrawn from a second vial into the syringe barrel, and mixing the first fluid and the second fluid in the syringe barrel.

In some embodiments of the invention, the hypodermic needle is inserted into a subject automatically

In some embodiments of the invention, a sleeve slidably covers said hypodermic needle following removal of the syringe extension from said subject.

According to a still further aspect of some embodiments of the present invention there is provided an extension assembly for a syringe barrel, the assembly including: a housing having a side wall, a first end and a second end. The assembly additionally includes a coupling extending from the first end, the coupling configured to attach to a syringe barrel. The assembly further includes a curved fluid channel extending from the coupling to a filling port, a filling port extending from the side wall, the filling port configured to receive a vial such that when the vial is assembled in the filling port, the vial is selectively in fluid communication with the syringe barrel via the curved fluid channel. The assembly still further includes at least one resilient element located within the housing, and at least one needle bearing element adapted, when actuated, to be displaced by at least one resilient element with respect to the housing from a non-penetration position to a penetration position, and a needle guard adapted for positioning with respect to the housing.

In some embodiments of the invention, the rearward displacement of the needle guard is operative to actuate displacement of the at least one needle bearing element from the non-penetration position to the penetration position.

In some embodiments of the invention, the assembly includes a safety element adapted to prevent inadvertent actuation of displacement of the at least one needle bearing element.

In some embodiments of the invention, the safety element additionally prevents inadvertent rearward displacement of the needle guard.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 shows an exploded view of a syringe barrel extension having a filling port, according to some embodiments of the invention; and

FIGS. 2-8 show operation of the syringe barrel extension shown in FIG. 1, according to some embodiments of the invention;

FIGS. 9A-9B show partial cut-away views demonstrating details of a fluid valve of the syringe barrel extension shown in FIG. 1, according to some embodiments of the invention;

FIGS. 10A-10B show details of a fluid channel through the syringe barrel extension shown in FIG. 1, according to some embodiments of the invention; and

FIGS. 11-12 show schematic views demonstrating details of the fluid channel shown in FIGS. 10A-10B, according to some embodiments of the invention.

### DESCRIPTION OF SPECIFIC EMBODIMENTS OF THE INVENTION

The present invention, in some embodiments thereof, relates to a syringe barrel extension, more particularly, but not exclusively, to a syringe barrel extension including a rotatable curved fluid channel selectively extending between a syringe barrel, a filling port, and a needle.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practice or carried out in various ways.

Referring now to the drawings:

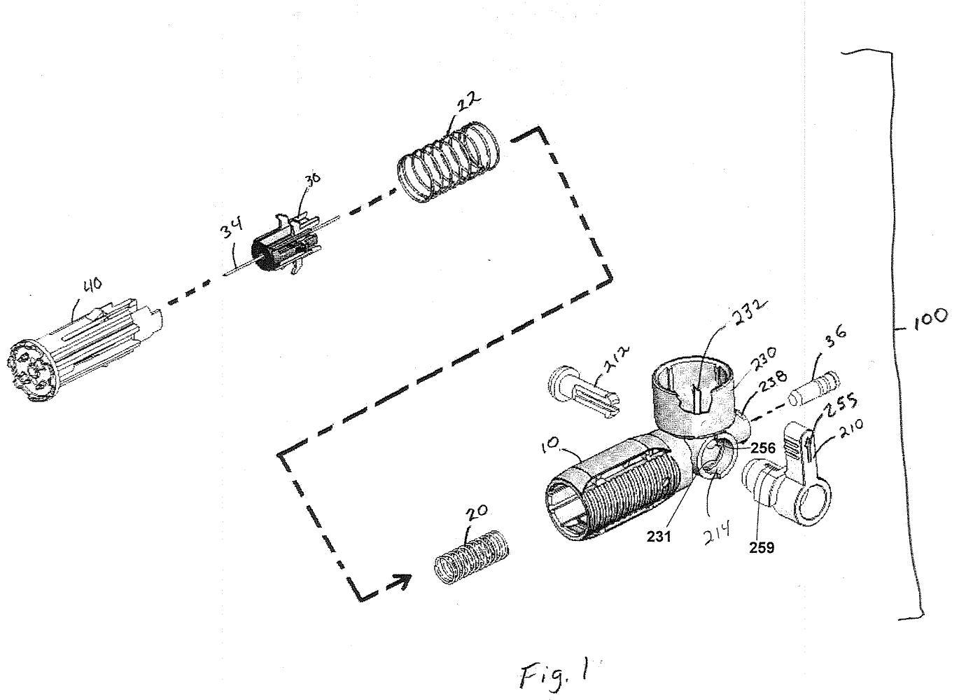

FIG. 1 shows an exploded view of a syringe barrel extension 100 comprising a housing 10 into which are generally coaxially seated respective first and second compression springs 20 and 22, which provide selectable forward displacement to a needle hub 30 and needle guard 40 respectively.

Needle hub 30 includes a needle 34 that extends forward into needle guard 40 and backward into housing 10.

Syringe barrel extension 100 includes a plug 36 that fits into housing 40 within a luer adapter 238. In embodiments, luer adapter 238 can be configured to be attached to any one of standard luer-slip, luer-lock plastic or glass syringe.

Additionally, luer adapter 238 may be optionally modified for engagement with various injection devices such as pen injectors.

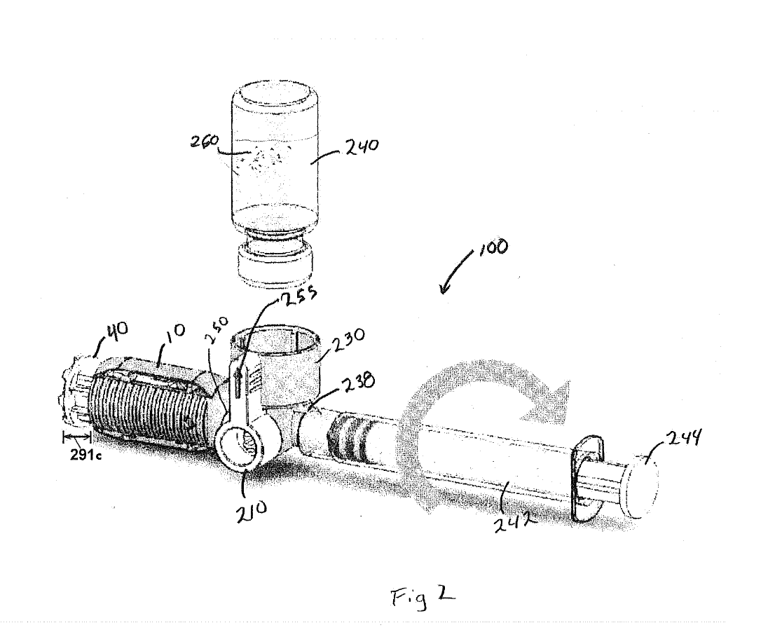

Syringe barrel extension 100 includes a fluid valve 210 that fits into a fluid valve port 214 and swivels with respect to housing 10 as will be explained below. Fluid valve 210 is held in place in fluid valve port 214 with a fluid valve clip 212 and provides fluid communication between a filling port 230 and a syringe barrel 242 (FIG. 2).

Filling port 230 is configured to receive a vial 240 (FIG. 2) which may typically contain medication 260 in liquid form, which is drawn into syringe barrel 242.

In some embodiments, syringe barrel 242 contains medication 260 in powdered form and drawing of medication 260 in fluid form from vial 240 allows mixture of the two medications 260 within syringe barrel 242.

In still further optional embodiments, a first vial 240 containing first medication 260 in fluid form may be attached to filling port 230 and first medication 260 is withdrawn into syringe barrel 242. Following this, a second vial 240 containing second medication 260 in fluid form is withdrawn into syringe barrel 242, thereby allowing mixture of first and second medications 260 within syringe barrel 242.

The many options for medication 260 comprising a fluid or powder in syringe barrel 242 and/or vial 240 are well-known to those familiar with the art.

It will be appreciated by those skilled in the art that compression springs 20 and 22 may be optionally replaced or enhanced by any material having plastic, resilient, and/or elastomeric properties in tension and/or compression.

FIG. 2 shows syringe barrel 242 being attached to luer adapter 238. While syringe barrel extension 100 is shown as separate from syringe barrel 242, it is also possible to integrate syringe barrel extension 100 with syringe barrel 242, or another injection device, and thus to eliminate the need for luer adapter 238.

As used herein, the term syringe refers to an apparatus of metal, glass, or plastic material consisting of a nozzle, or needle, barrel, and plunger or rubber bulb; used to inject a fluid into a cavity or under the skin. The term syringe a priori includes any type of apparatus used to inject a fluid into a cavity or under the skin, inter alia, an asepto syringe, a bulb syringe, a hypodermic syringe, and a luer-lock syringe.

FIG. 3 shows vial 240 containing medication 260 in fluid form, being attached to filling port 230. Filling port 230 includes a hollow tube 232 that pierces through a typical rubber covering on vial 240 containing medication 260 in fluid form, as described above.

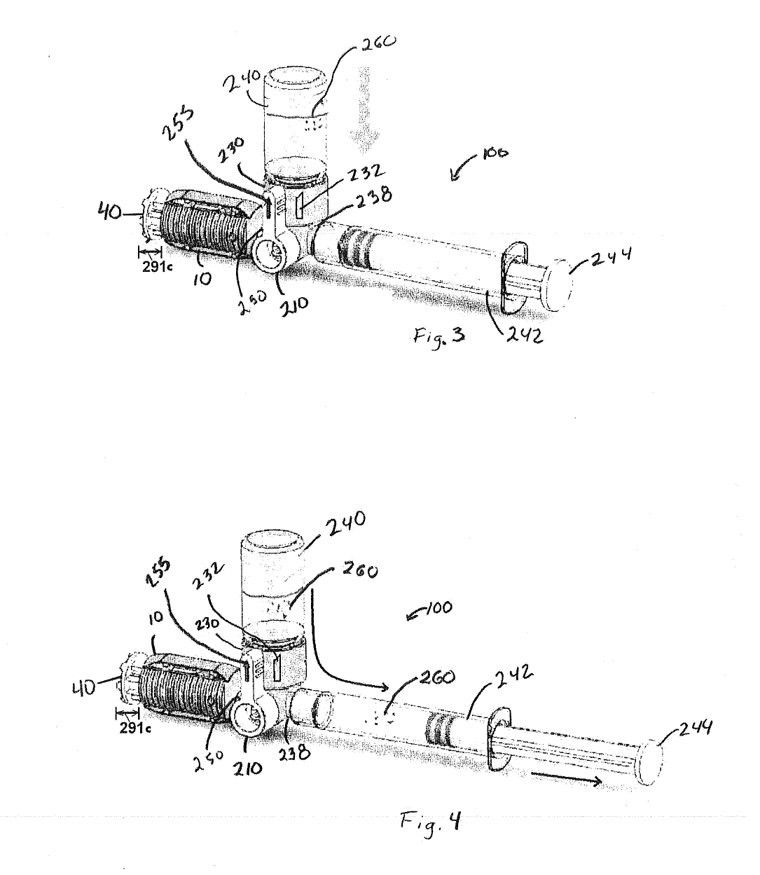

FIG. 4 shows a syringe plunger 244 being withdrawn from syringe barrel 242, thereby causing medication 260 in fluid form in vial 240 to enter syringe barrel 242.

While vial 240 is shown attached to filling port 230 following attachment of syringe barrel 242 to luer adapter 238, alternatively vial 240 is optionally attached to filling port 230 prior to the attachment of syringe barrel 242 and therefore allowing venting of vial 240.

In one optional method for venting, syringe barrel 242 is partially filled with air prior to attachment to luer adapter 238, with the air being injected into vial 240, thereby possibly allowing freer movement of the fluid into syringe barrel 242 upon withdrawal of syringe plunger 244.

As shown in FIG. 5, following filling of syringe barrel 242, vial 240 is optionally removed from filling port 230.

In some embodiments of the invention, syringe barrel 242 may have a larger volume to accommodate more than one medication 260, thereby allowing mixing of medications 260 as noted above.

In such instances first vial 240 may contain a fluid and second vial 240 (not shown) may contain drug in powder or liquid form to be mixed in the second vial 240. In such cases, the syringe is filled with the first fluid and the contents are then ejected into the second vial. The mixture from the second vial is then withdrawn into syringe barrel 242 prior to being injected into a user.

Optionally vial 240 may be left in place in filling port 230 during the subsequent steps of injection, explained below.

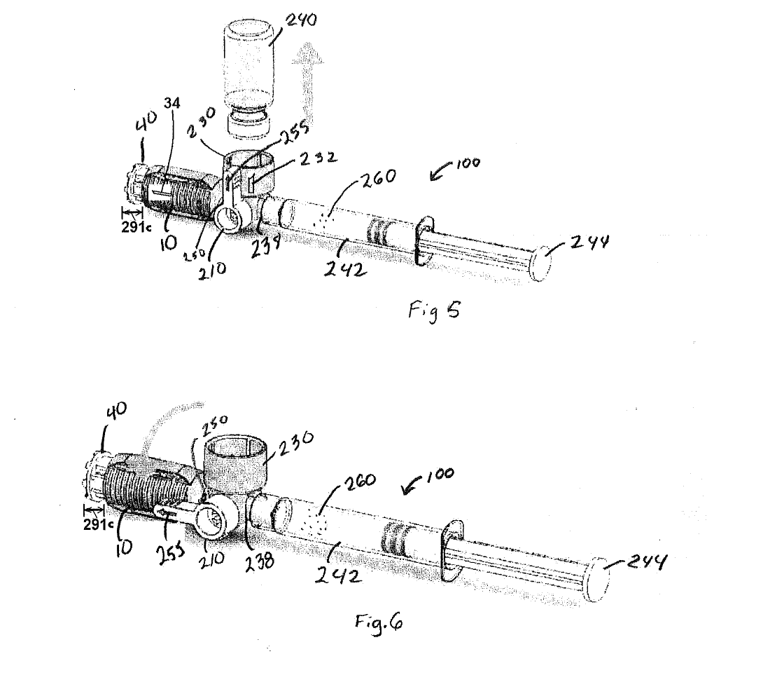

As shown in FIG. 6, fluid valve 210 is rotated from the vertical to horizontal position thereby allowing dispensing of medication 260 contained within syringe barrel 242 through needle 34.

FIG. 7 shows syringe barrel extension 100 pressed against a portion of tissue 262, for example of a human, such that needle guard 40 retracts into housing 10.

Additionally, needle hub assembly 30 with spring 20, seen in partial cross section, is automatically released by the movement of needle guard 40, thereby causing needle hub assembly 30 to be automatically propelled axially forward by the above-noted spring, thereby causing needle 34 to extend and automatically penetrate portion of tissue 262; as described in above-noted US Patent Application 20070118081.

Retraction of needle guard 40 assumes a compressed distance 291a which is less than distance 291c as shown in FIGS. 2-6; as additionally described in above-noted US Patent Application 20070118081.

With needle 34 positioned in tissue 262, syringe plunger 244 is pressed towards syringe barrel 242 thereby causing medication 260 to be deposited within tissue 262.

Optionally, needle 34 may be of one of several lengths to allow deposit of medication to any one of several depths within tissue 262. For example, injection into the abdominal area of an obese person may require a longer needle 34 than injection into the arm of an infant. Additionally, intra muscular, intradermal and subcutaneous injections are often performed with needles of different length and/or bore size.

Additionally or alternatively, needle 34 may have one of several gauges to allow faster or slower deposit of medication 260 in tissue 262.

Optionally needle guard 40 is color coded according to the length and/or gauge of needle 34 which is contained within syringe barrel extension 100.

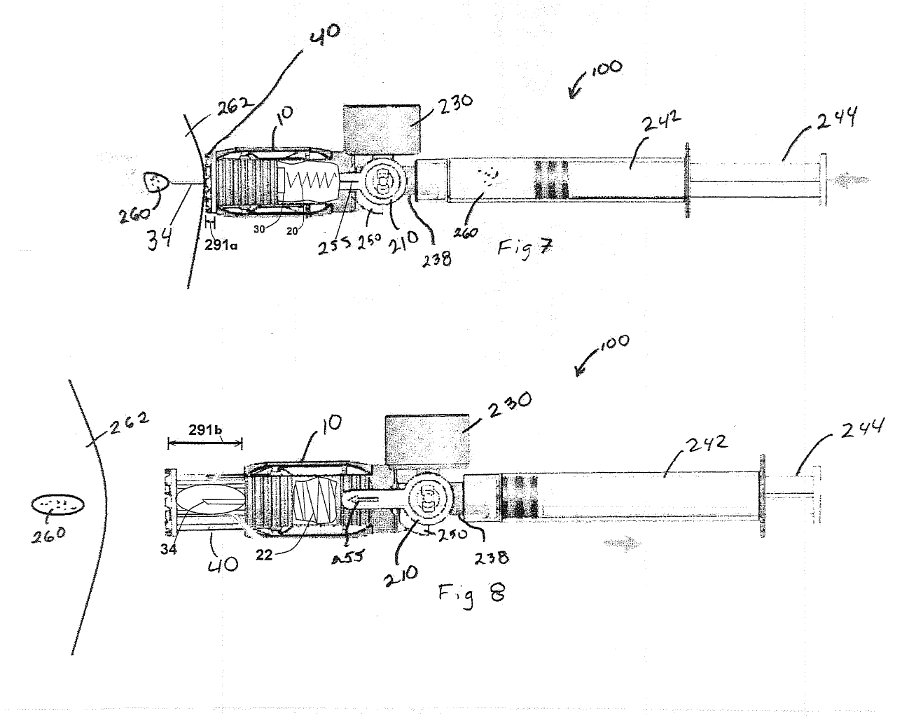

FIG. 8 shows syringe barrel extension 100 removed from tissue 262 following injection of the user.

As the pressure against needle guard 40 is released, needle guard 40 moves axially forward, to fully extend distance 291b which is greater than distance 291c as shown in FIGS. 2-6; and described in above-noted US Patent Application 20070118081.

At the above noted greater distance 291b, (FIG. 8), inadvertent needle pricks are prevented; as described in above-noted US Patent Application 20070118081.

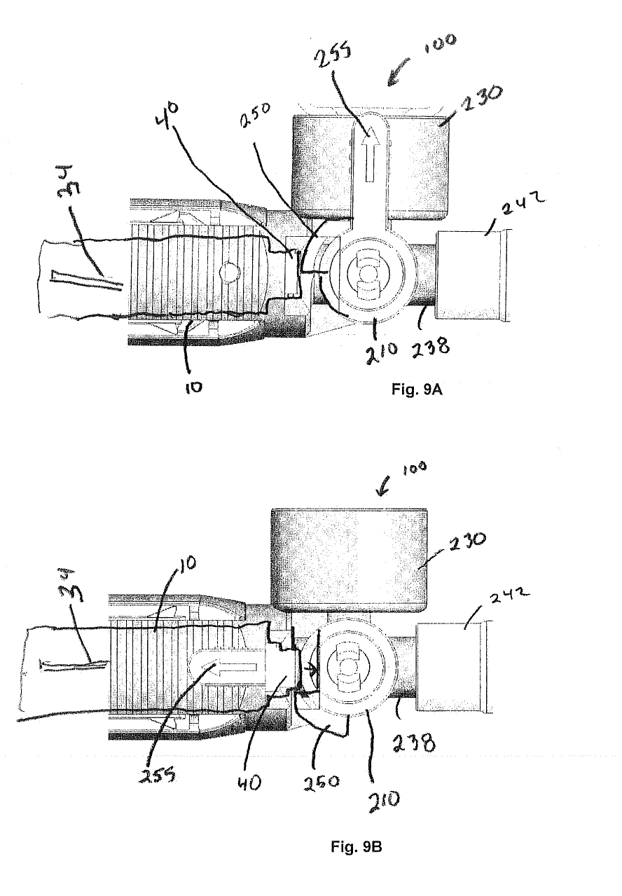

FIGS. 9A-9B show details of the interaction between a needle stop 250 and needle guard 40. When fluid valve 210 is in the vertical portion (FIG. 9A) needle stop 250 rests against the rear portion of needle guard 40, thereby preventing inadvertent movement of needle guard 40 that could allow a user to be pricked by needle 34.

With fluid valve 210 rotated to the horizontal position (FIG. 9B) needle stop 250 moves below the rear portion of needle guard 40 thereby allowing movement of needle guard 40 in a rearward position that allows exposure of needle 34 as shown in FIG. 7.

Additionally, to aid a user in filling and dispensing medication, fluid valve 210 includes an arrow 255 that points up toward vial 240 during the filling of syringe barrel 242; thereby indicating that vial 240 is in fluid communication with syringe barrel 242.

When fluid valve 210 is rotated toward needle 34, arrow 255 points toward needle 34, thereby indicating that syringe barrel 242 is in fluid communication with needle 34.

While the indicator of the position of fluid valve 210 is shown as arrow 255, a variety of indicator shapes and colors may be used to indicate the direction of fluid flow, including colored dots, and rectangular or curvilinear shapes.

To direct fluid from port 230 to syringe barrel 242, and then to direct fluid from syringe barrel 242 to needle 34, there are many valve options. Just one of the many options for such a valve including the selectively extendable curved fluid channel which will now be described.

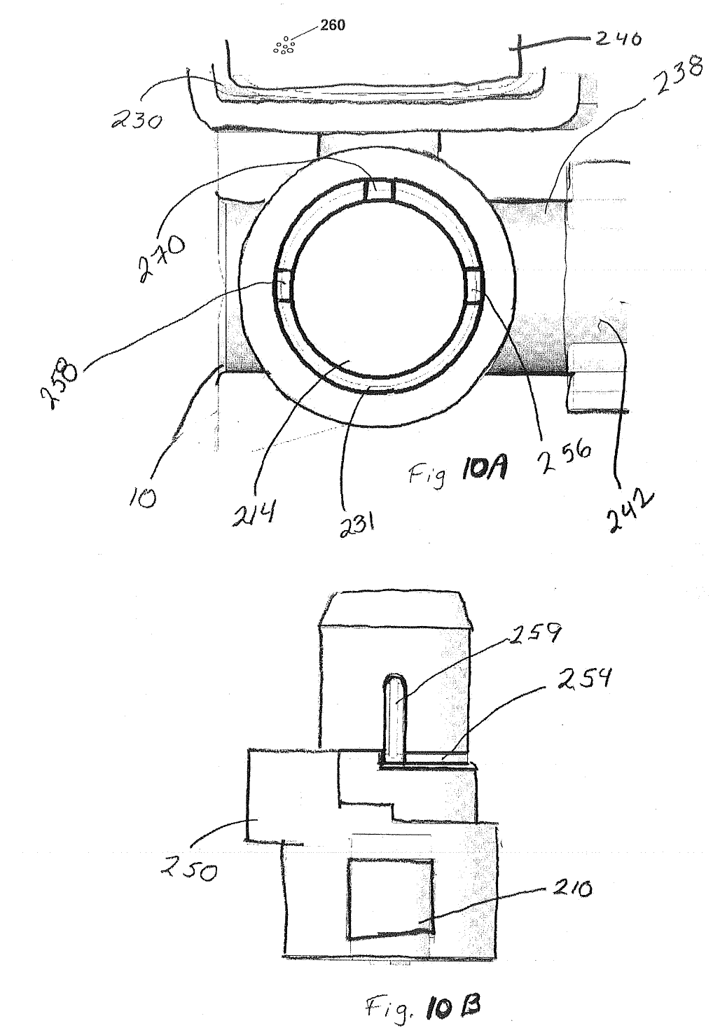

FIG. 10A shows details of a port trough 231 along a perimeter within fluid valve port 214. Port trough 231 includes syringe channel 256 which is connected to luer adapter 238. Port channel 270 is connected to hollow tube 232 of filling port 230 and needle channel 258 is connected to needle 34. Port channel 270 and needle channel 258 are not, at this stage, in fluid connection, nor are port channel 270 and needle channel 258 in fluid connection with port trough 231.

FIG. 10B shows details of a valve trough 254, on the external surface of fluid valve 210, which includes a valve channel 259.

Port trough 231 (FIG. 10A) and valve trough 254 slidingly move past each other during movement of fluid valve 210 such that valve trough 254 has a variable extension during rotation of fluid valve 210, and attains fluid communication with syringe channel 256.

Initially, valve channel 259 aligns with port channel 270 (FIG. 10A) to allow fluid communication between hollow tube 232 of filling port 230 and syringe barrel 242 (FIG. 10A).

Following rotation, valve channel 259 aligns with needle channel 258 (FIG. 10A) allow syringe barrel 242 (FIG. 10A) to be in fluid communication with needle 34, while fluid entering the lower portion of port trough 231 (FIG. 10A) reaches a dead-end.

As used herein, the term “fluid communication” means communication between a gas or liquid in vial 240 and a gas or liquid in syringe barrel 242. It should be understood that vial 240 and/or syringe barrel may contain a gas such as air plus medication, inter alia, in liquid, gel, or powdered form.

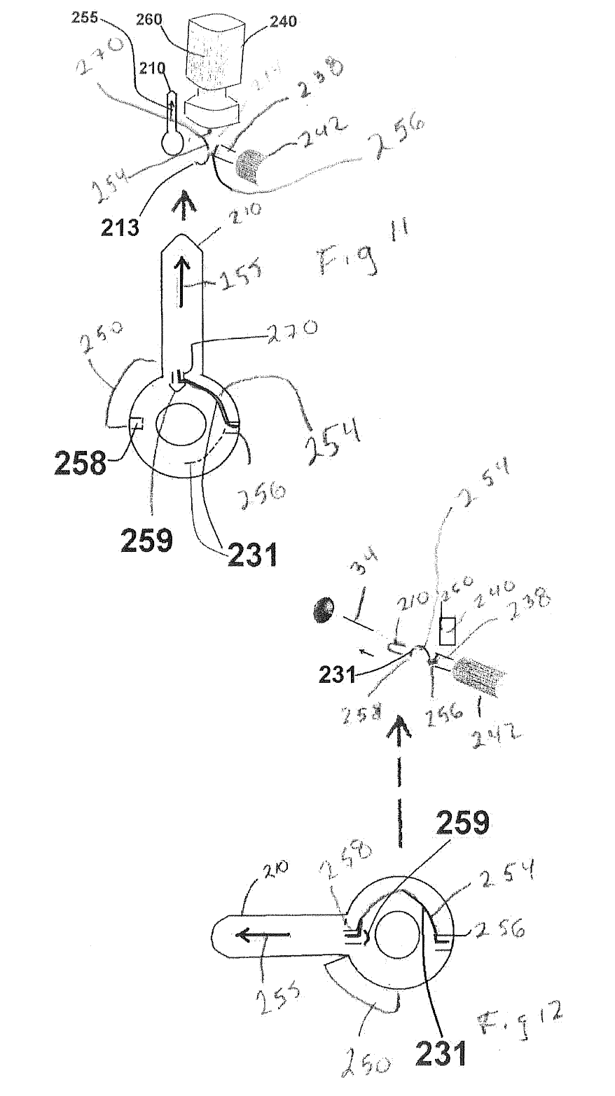

FIGS. 11-12 show details of the interaction between curved valve fluid channel 254 and curved port fluid channel 231.

As noted above, with fluid valve 210 in the vertical portion, (FIG. 11) curved valve channel 259 communicates with syringe channel 256 via fluid port channel 270; thereby allowing fluid to flow between vial 240 and syringe barrel 242.

With fluid valve 210 in the horizontal position (FIG. 12), as noted above, curved valve channel 259 extends from syringe channel 256 to needle channel 258; thereby allowing fluid to flow between syringe barrel 242 and through needle 34.

It is expected that during the life of a patent maturing from this application many relevant hypodermic syringe devices having extendable needles will be developed and the scope of the phrase hypodermic syringe devices having extendable needles is intended to include all such new technologies a priori.

As used herein the term “about” refers to ±10%.

The terms “comprises”, “comprising”, “includes”, “including”, “having”, and their conjugates mean “including but not limited to”.

The term “consisting of” means “including and limited to”.

The term “consisting essentially of” means that the composition, method or structure may include additional ingredients, steps, and/or parts, but only if the additional ingredients, steps, and/or parts do not materially alter the basic and novel characteristics of the claimed composition, method, or structure.

As used herein, the singular form “a”, “an”, and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6, etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques, and procedures for accomplishing a given task including, but not limited to, those manners, means, techniques, and procedures either known to, or readily developed from known manners, means, techniques, and procedures by practitioners of the chemical, pharmacological, biological, biochemical, and medical arts.

As used herein, the term “treating” includes abrogating, substantially inhibiting, slowing, or reversing the progression of a condition, substantially ameliorating clinical or aesthetical symptoms of a condition, or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination, or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications, and variations will be apparent to those skilled in the art. Accordingly, it is intended to embrace all such alternatives, modifications, and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1-31. (canceled)

32. An extension assembly for a syringe barrel, said assembly comprising:
i) a housing having a side wall, a first end and a second end;
ii) a coupling extending from said first end, said coupling configured to attach to a syringe barrel;
iii) a fluid channel extending from said coupling to a filling port; and
iv) a filling port extending from said side wall, said filling port configured to receive a vial which is selectively in fluid communication with said syringe barrel via said fluid channel;
v) at least one first resilient element associated with said housing, and at least one needle bearing element configured to be displaced by said at least one first resilient element with respect to said housing element from a non-penetration position to a penetration position;
vi) a fluid valve having at least two positions:
at least one first position in which said vial receiving port is in fluid communication with said syringe barrel; and
at least one second position in which said needle bearing element is in fluid communication with said syringe barrel.

33. The assembly according to claim 32, wherein said coupling is configured to attach to said syringe barrel in at least one of the following manners:
i) removably; and
ii) irremovably.

34. The assembly according to claim 32, wherein said filling port extends at about 90 degrees to the longitudinal axis of said housing.

35. The assembly according to claim 32, comprising a slidable needle guard operatively surrounding and guarding said needle in a non-penetration arrangement, when said valve is in said first position; wherein in said second valve position said slidable needle guard is retractable to expose said needle-bearing element to a penetration position.

36. The assembly according to claim 35, wherein said slidable needle guard retracts or extends by sliding coaxially along said housing, wherein in said retracted position said needle guard may be present either externally or internally relative to said housing.

37. The assembly according to claim 32, comprising a moveable needle stop for preventing inadvertent exposure of said needle bearing element.

38. The assembly according to claim 37, wherein in said first position in which said vial receiving port is in fluid communication with said syringe barrel, said needle stop rests against an internal portion of a needle guard and prevents movement of said needle guard.

39. The assembly according to claim 35, wherein resilient elements are biased to propel said slidable needle guard axially to a fully extended position beyond the penetrating end of said needle, to prevent inadvertent needle pricks after use of said assembly.

40. The assembly according to claim 39, wherein a releasable locking element secures said slidable needle guard in said fully extended position.

41. The assembly according to claim 32, wherein visual indicia are present upon an external side of said valve, to indicate to a user which of said positions said valve is in.

42. The assembly according to claim 32, wherein said filling port includes a vial-piercing member configured to pierce a vial such that said piercing facilitates said fluid communication with a syringe barrel.

43. The assembly according to claim 35, wherein rearward displacement of said needle guard is operative to actuate displacement of said at least one needle bearing element from said non-penetration position to said penetration position.

44. The assembly according to claim 37, wherein said needle stop additionally prevents inadvertent rearward displacement of a needle guard.

45. A kit for injection of a medicament, comprising:
a) the extension assembly of claim 32;
b) a syringe;
c) a vial filled with a medicament.
